(19) United States
(12) Patent Application Publication
(10) Pub. No.: US 2013/0018450 A1
(43) Pub. Date: Jan. 17, 2013

(54) PROSTHESIS DELIVERY SYSTEM WITH RETENTION SLEEVE

(71) Applicant: James B. Hunt, Bloomington, IN (US)

(72) Inventor: James B. Hunt, Bloomington, IN (US)

(21) Appl. No.: 13/182,014

(22) Filed: Jul. 13, 2011

(51) Int. Cl. A61F 2/84

(52) U.S. Cl. 623/1.12

(57) ABSTRACT

A system for open surgical repair of a body vessel is described herein. A retention sleeve receives an expandable prosthesis. The sleeve has a delivery and a deployed configuration. In the delivery configuration, the sleeve has at least one overlapped region and the sleeve is sized to retain the prosthesis in a compressed configuration for insertion of ends of the prosthesis into the vessel. In the deployed configuration, the sleeve is moves to a larger cross-sectional area to allow for expansion of the ends of the prosthesis for engagement with the vessel. One or more releasable members are extendable through the overlapped region to retain the sleeve in the delivery configuration. The releasable member is removable from the overlapped region, preferably from the center of the prosthesis, to permit the sleeve to move to the deployed configuration and expansion of the outer ends prior to the center of the prosthesis.

### BACKGROUND

The present disclosure relates generally to medical devices for emergency repair of body vessels. More particularly, it relates to prosthesis delivery systems used for repairing damaged body vessels and gaining hemostasis during emergency open surgical procedures.

Trauma physicians frequently encounter patients having traumatic injury to a body vessel, such as lacerated vessels or even transected vessels, resulting from gunshots, knife wounds, motor vehicle accidents, explosions, etc. Significant damage to a body vessel may expose a patient to deleterious conditions such as the loss of a limb, loss of function of a limb, increased risk of stroke, impairment of neurological functions, and compartment syndrome, among others. Particularly, severe cases of vascular injury and blood loss may even result in death. In such severe situations, the immediate goal is to obtain hemostasis while maintaining perfusion of adequate blood flow to critical organs, such as the brain, liver, kidneys, and heart.

Examples of treatment that are commonly performed by trauma physicians to treat body vessel injuries include the clamping of the vessel with a hemostat, the use of a balloon tamponade, the ligation of the damaged vessel at or near the site of injury, or the insertion of one or more temporary shunts. However, conventional surgical repair is generally difficult with such actively bleeding, moribund patients. In many instances, there is simply not enough time to repair the body vessel adequately by re-approximating and suturing the body vessel. In many situations, the trauma physician will simply insert a temporary shunt (such as a Pruitt-Inahara Shunt) into the vessel. However, use of temporary shunts has been linked to the formation of clots. This may require returning the patient to the operating room for treatment and removal of the clots, often within about 36 to 48 hours of the original repair. Since shunts are generally placed as a temporary measure to restore blood flow and stop excessive blood loss, the shunt is typically removed when the patient has stabilized (generally a few days later) by a specialized vascular surgeon. After removal, the vascular surgeon will replace the shunt with a vascular graft, such as a fabric graft that is sewn into place. With respect to ligation, ligation of the damaged blood vessel may result in muscle necrosis, loss of muscle function, or a potential limb loss or death.

Due to the nature of the body vessel injury that may be encountered, the insertion of shunts or ligation of a blood vessel, for example, often requires that such treatments be rapidly performed at great speed, and with a high degree of physician skill. Such treatments may occupy an undue amount of time and attention of the trauma physician at a time when other pressing issues regarding the patient's treatment require immediate attention. In addition, the level of particularized skill required to address a vascular trauma may exceed that possessed by the typical trauma physician. Particularly, traumatic episodes to the vessel may require the skills of a physician specially trained to address the particular vascular trauma, and to stabilize the patient in the best manner possible under the circumstances of the case.

Some open surgical techniques utilize sutures to affix damaged tissue portions surrounding fittings that have been deployed with the vessel, which requires the trauma physician to take time to tie the sutures properly. Although in modern medicine sutures can be tied in relatively rapid fashion, any step in a repair process that occupies physician time in an emergency situation is potentially problematic. In addition, the use of sutures to affix the vessel to the fitting compresses the tissue of the vessel against the fitting. Compression of tissue may increase the risk of necrosis of the portion of the vessel tissue on the side of the suture remote from the blood supply. When present, necrosis of this portion of the vessel tissue may result in the tissue separating at the point of the sutures. In this event, the connection between the vessel and the fitting may eventually become weakened and subject to failure. If the connection fails, the device may disengage from the vessel. Therefore, efforts continue to develop techniques that reduce the physician time required for such techniques, so that this time can be spent on other potentially life-saving measures, and the blood flow is more quickly restored and damage caused by lack of blood flow is minimized.

Trauma physicians generally find it difficult to manipulate a prosthesis for insertion into a body vessel that has been traumatically injured. For example, one difficulty arises from the trauma physician trying to limit the size of the opening created for gaining access to the injured vessel so that such opening requiring healing is as small as possible. Another difficulty is that the injured vessel can be anywhere in the body, having different surrounding environments of bone structure, muscle tissue, blood vessels, and the like, which makes such obstructions difficult to predict in every situation and leaves the trauma physician working with an even further limited access opening. Another potential consideration is the amount of body vessel removed during a transection. The goal would be to remove a portion of the body vessel as small as possible. Yet, a small portion removed from the vessel leaves such a small space between the two vessel portions, thereby making it difficult to introduce the prosthesis between the two vessel portions.

Thus, what is needed is a prosthesis delivery system for use in open surgical repair of an injured body vessel, such as an artery or a vein, (and in particular a transected vessel) during emergency surgery. It would be desirable if such prosthesis delivery system is easy for a trauma physician to use, and can be rapidly introduced into two vessel portions of a transected vessel, thereby providing a conduit for blood within the injured body vessel.

### SUMMARY

Accordingly, in one embodiment a system is provided herein to address at least some of the shortcomings of the prior art. The system can be used to interconnect two vessel portions such as for open surgical repair of a transected body vessel. The system includes a sleeve member conformable into a tubular body having a passageway extending therethrough for receiving a prosthesis. The prosthesis is movable between a compressed configuration and an expanded configuration. The sleeve member is movable between a delivery configuration and a deployed configuration. In the delivery configuration, the sleeve member can have at least one overlapped region, such as a fold or overlapped edges. Further, the passageway has a first cross-sectional area sized to retain the prosthesis in the compressed configuration for insertion into a body vessel. In the deployed configuration, the passageway increases to a second cross-sectional area greater than the first cross-sectional area to allow for expansion of the prosthesis to the expanded configuration for engagement with the body vessel. The system also includes at least one releasable member that extends through the overlapped region of the sleeve member to retain the sleeve member in the delivery configuration. The releasable member is removable from the overlapped region to permit the sleeve member to move the deployed configuration.

In one aspect, the retention sleeve is movable between a first configuration and a second configuration. In the first configuration, the retention sleeve has at least one overlapped region and the sleeve passageway is sized to retain the prosthesis in the compressed configuration for insertion into a body vessel. In the second configuration, the sleeve passageway has a larger cross-sectional area to allow for expansion of the prosthesis to the expanded configuration for engagement with the body vessel. A first releasable wire member can extend through a first length of the overlapped region of the retention sleeve from the first outer end to the intermediate region of the prosthesis. A second releasable wire member can extend through a second length of the overlapped region of the retention sleeve from the second outer end to the intermediate region of the prosthesis. In response to removal of the first releasable wire member from the first length of the overlapped region of the retention sleeve in a first outside-in direction at the intermediate region of the prosthesis, the first outer end of the prosthesis is allowed to expand. In response to removal of the second releasable wire member from the second length of the overlapped region of the retention sleeve in a second outside-in direction, opposite the first outside-in direction, at the intermediate region of the prosthesis, the second outer end of the prosthesis is allowed to expand.

In another embodiment, a method of open surgical repair of a body vessel having a first vessel portion and a second vessel portion is provided. The method can include one or more of the following steps, such as introducing a first outer end of a prosthesis into a first vessel portion. The prosthesis is retained in a compressed configuration by a sleeve member having an overlapped configuration. The sleeve member is maintained in the overlapped configuration with a releasable member extending through an overlapped region. The first outer end of the prosthesis is retained in the compressed configuration by a first segment of the sleeve member in the overlapped configuration. The releasable member can be removed from the overlapped region of the sleeve member to permit movement of the first segment of the sleeve member to a larger cross-sectional area such that the first outer end of the prosthesis is permitted to expand for engagement with a wall of the first vessel portion. A second outer end of the prosthesis may be introduced into a second vessel portion. The second outer end is retained in the compressed configuration by a second segment of the sleeve member in the overlapped configuration. The releasable member, the same as the one in the first segment or a different one, is removed from the overlapped region of the sleeve member. The second segment of the sleeve member is then permitted to move to a larger cross-sectional area such that the second outer end of the prosthesis is permitted to expand for engagement with a wall of the second vessel portion.

### BRIEF DESCRIPTIONS OF THE DRAWINGS

FIG. 1A is a perspective view of one example of a prosthesis delivery system with a retention sleeve in a delivery configuration.

FIG. 1B is a perspective view of the prosthesis delivery system of FIG. 1A with the retention sleeve in a deployed configuration.

FIG. 2A is a perspective view of one example of a prosthesis delivery system with a retention sleeve in a delivery configuration.

FIG. 2B is a perspective view of the prosthesis delivery system of FIG. 2A with the retention sleeve in a deployed configuration.

FIG. 3 is a transverse sectional view of the prosthesis delivery system of FIG. 1A.

FIG. 4 is a perspective view of another example of a prosthesis delivery system with a removable retention sleeve.

FIG. 5 is a transverse sectional view of a prosthesis delivery system with a plurality of overlapped regions and releasable wires.

FIG. 6 is a perspective view of an end of a prosthesis delivery system with apertures formed in a retention sleeve.

FIGS. 7A-7B are perspective views of an end of a prosthesis delivery system with a retention sleeve with evertable ends.

FIG. 8 is a perspective view of another example of a prosthesis delivery system with a retention sleeve sized shorter than a prosthesis.

FIG. 9A is a perspective view of another example of a prosthesis delivery system with a graft body of a prosthesis forming the retention sleeve along the exterior of the prosthesis.

FIGS. 9B-9C are transverse sectional views of the prosthesis delivery system of FIG. 9A, depicting movement between delivery and deployed configurations.

FIG. 10A is a perspective view of another example of a prosthesis delivery system with a graft body of a prosthesis forming the retention sleeve along the interior of the prosthesis.

FIGS. 10B-10C are transverse sectional views of the prosthesis delivery system of FIG. 10A, depicting movement between delivery and deployed configurations.

FIGS. 11A-11F illustrate a method of open surgical repair of two body vessel portions with one example of a prosthesis delivery system.

### DETAILED DESCRIPTION OF THE DRAWINGS AND THE PRESENTLY PREFERRED EMBODIMENTS

For the purposes of promoting an understanding of the principles of the present disclosure, reference will now be made to the embodiments illustrated in the drawings, and specific language will be used to describe the same. The prosthesis delivery system described herein can be useful for open surgical repair of a body vessel, such as a blood vessel, during an emergency procedure. This prosthesis delivery system can be particularly useful for repair of a lacerated artery or vein during emergency surgery, and particularly, to obtain hemostasis while maintaining blood perfusion. Other applications for the prosthesis delivery system will become readily apparent to one skilled in the art from the detailed description.

The prosthesis delivery system described herein can deploy a prosthesis that is useful for repair of vessels, lumens, ducts, or passageways of the body, with the term “body vessel” used in the specification to describe theses structures in general, during emergency open surgical repair. The prosthesis delivery systems described herein can include a retention sleeve fitted over a prosthesis. The retention sleeve can be movable between a first, delivery configuration and a second, deployed configuration. In the delivery configuration, the retention sleeve can include one or more overlapped regions so that the retention sleeve defines a first cross-sectional area sized to retain the prosthesis in a radially compressed configuration for insertion into a body vessel opening. One or more releasable members can be inserted through the overlapped region in order to maintain different segments of the retention sleeve in the delivery configuration. Removal of a releasable member from the overlapped region can permit the corresponding segment of the retention sleeve to move to the deployed configuration. In the deployed configuration, the overlapped region becomes non-overlapped or the fold is unfolded and the retention sleeve increases in size to a second cross-sectional area sufficient to permit different regions of the prosthesis to expand in a radially expanded configuration for engagement with the body vessel wall.

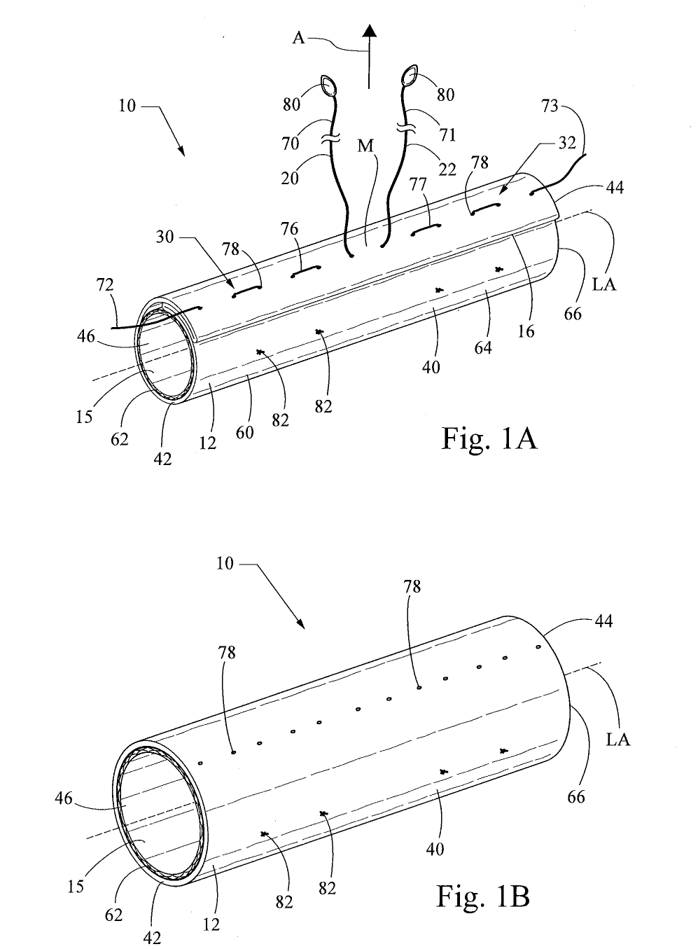

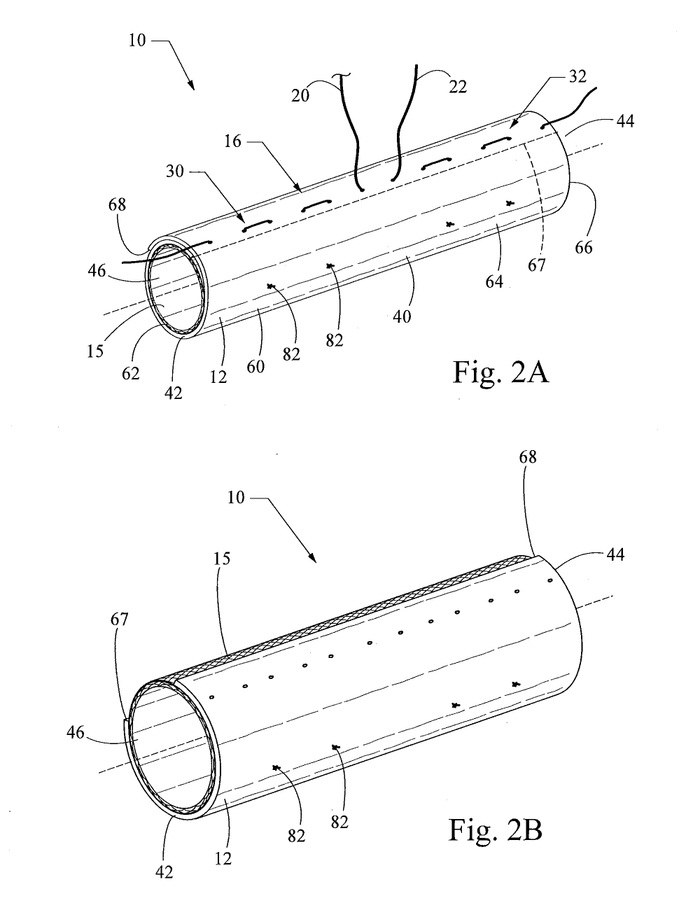

FIGS. 1A and 2A depict embodiments of a prosthesis delivery system 10 having a retention sleeve 12 in a delivery, overlapped configuration fitted over a prosthesis 15 in a compressed configuration. The retention sleeve 12 can include an overlapped region 16 extending in a longitudinal direction along a longitudinal axis LA of the prosthesis. The overlapped region 16 can be composed of a fold or pleat (FIG. 1A) in the case of an integral sleeve or a first edge in an overlapping relationship with a second edge (FIG. 2A) in the case of a sleeve that is pre-split. A first releasable member 20 and a second releasable member 22 are extendable through a first length 30 and a second length 32 of the overlapped region 16 in a manner to maintain the respective lengths of the overlapped region 16 in the retention sleeve 12. As a result, corresponding segments of the retention sleeve 12 are maintained in the delivery configuration. FIGS. 1B and 2B depict the delivery system 10 with the retention sleeve 12 in a deployed, non-overlapped configuration and the prosthesis 15 in an expanded configuration as a result of the removal of the first and second releasable members 20, 22 from the overlapped region 16.

The retention sleeve 12 can include a tubular body 40 that extends between a first sleeve end 42 and a second sleeve end 44. A passageway 46 extends through the tubular body 40 and is sized to receive the prosthesis 15 in the compressed configuration. The overlapped region 16 can have a longitudinal configuration that extends at least partially between the first and second sleeve ends 42, 44. The retention sleeve 12 with the overlapped region 16 provides a reduction in the cross-sectional area of the passageway 46 of the retention sleeve 12 to a size so that, when the prosthesis 15 is in the compressed configuration, at least the outer end of the prosthesis can fit within the body vessel.

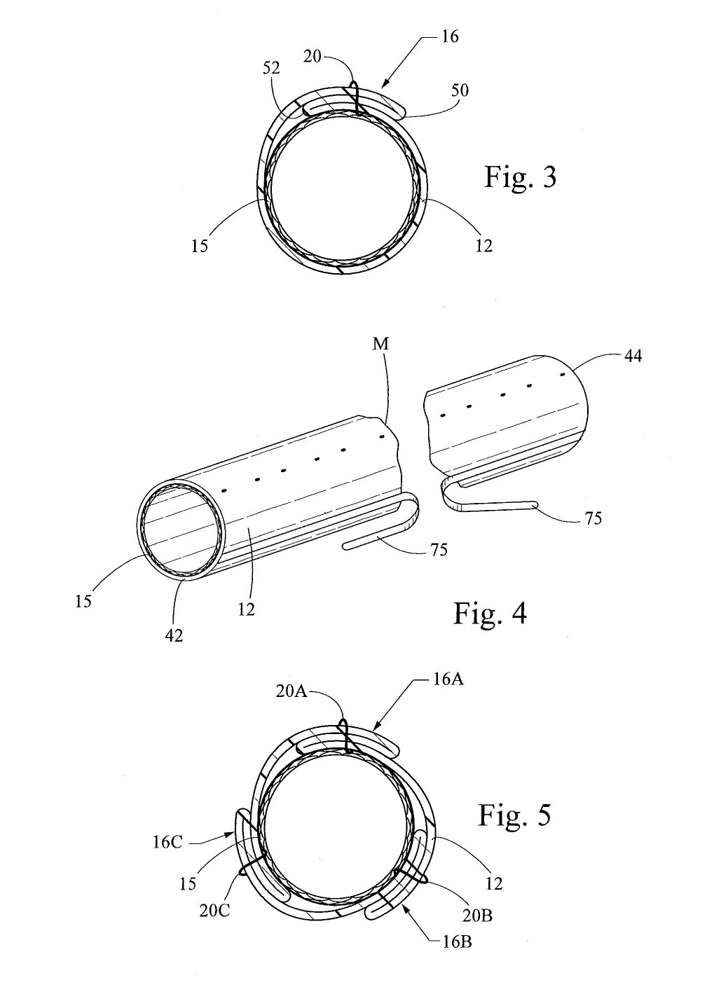

The releasable member can be coupled with the retention sleeve 12 in a manner to selectively maintain the overlapped region for delivery of the prosthesis into the body vessel. For example, the releasable members 20, 22 can be threaded or woven through the retention sleeve in an in-and-out configuration. In FIG. 3, the releasable member 20 can be threaded through the retention sleeve 12 closer to the tip 50 of the overlapped region as a fold than the base 52 of the overlapped region and through the retention sleeve so that the overlapped region 16 is positioned to lie flat against the retention sleeve. In another example, the releasable member can be threaded through the retention sleeve at the base of the overlapped region so that the overlapped region is extendable radially outward from the prosthesis. To reduce the delivery profile of the system, the end user can force the overlapped region to lie against the retention sleeve during insertion of the system into the body vessel. It is contemplated that the releasable member can be threaded through other portions of the overlapped region between the tip 50 and the base 52 and through the retention sleeve in order to accomplish the configurations described herein. It is contemplated that the releasable members may include one or more clamping elements, instead of or in addition to being threaded through the retention sleeve. For example, the clamping elements can include C-shaped members or ring members that fit around the retention sleeve and maintain the overlapped region in the retention sleeve. Retraction of the releasable member can remove the clamping element from its position around the overlapped region to release the overlapped region.

In FIGS. 1A and 2A, the first releasable member 20 can be threaded through the first length 30 of the overlapped region 16 corresponding to a first segment 60 of the retention sleeve 12, which is associated with a first outer end 62 of the prosthesis 15. The second releasable member 22 can be threaded through the second length 32 of the overlapped region 16 corresponding to a second segment 64 of the retention sleeve 12, which is associated with a second, opposite outer end 66 of the prosthesis 15. This arrangement can allow independent expansion of the outer ends 62, 66 of the prosthesis 15 prior to a middle M of the prosthesis 15 when the respective releasable members are removed. Other releasable members may be provided with the system to permit for expansion of different longitudinal and/or circumferential portions of the prosthesis if so desired.

FIGS. 2A-2B show the overlapped region 16 formed as a first edge 67 tucked under a second edge 68 to define an overlapping relationship therebetween. In FIG. 2B, after expansion of the prosthesis 15, the edges 67, 68 may be separated from one another, although the edges can be in an abutting relationship or still in an overlapping relationship. The retention sleeve configured with the first and second edges, i.e., pre-split, may allow for easier removal from the prosthesis after expansion.

According to FIG. 1A, each of the releasable members 20, 22 can include a respective first end portion 70, 71 that extends outwardly from the prosthesis at approximately the middle M of the prosthesis 15 and a respective second end portion 72, 73 that is proximate the outer ends, and may extend at least partially outwardly from the outer ends 62, 66 of the prosthesis 15. A respective intermediate portion 76, 77 of the releasable member disposed between the corresponding first and second end portions is the portion that is extended through the retention sleeve 12 to maintain the overlapped region 16. A series of openings 78 may be formed in the retention sleeve 12 for the passage of the releasable members 20, 22 therethrough. The openings 78 can be preformed prior to the releasable member being passed therethrough or can be formed by the releasable member during the threading process. To release the releasable members 20, 22 for expansion of the respective portion of the prosthesis 15, the end user can apply a retraction force to the respective first end portion 70, 71 at the middle M of the prosthesis 15 in an outward radial direction that is represented by arrow A. This can cause the releasable members 20, 22 each to move through the openings 78 in an outside-in direction. The size of the releasable member can be the same size as the opening, but preferably slightly smaller to facilitate the sliding action of the releasable member through the opening. The first end portions 70, 71 may have an enlarged end or portion 80 relative to the remaining portions of the releasable member to facilitate grippability and to increase the tensile strength of the wire member for retraction. The second end portions 72, 73 may have a reduced size relative to the remaining portions of the releasable member to reduce the possibility of snagging the retention sleeve or the openings when retracted. It is contemplated that the first end portions 70, 71 may be coupled to one another so that a single retraction of the coupled end portions can remove the releasable member from the sleeve. In one example, the releasable member with the coupled end portions 70, 71 is a single integral member with an intermediate bend portion disposed where the end portions are located.

In FIGS. 1B and 2B, once the releasable members are removed, the retention sleeve 12 can move to the deployed configuration so that the passageway 46 of the retention sleeve is enlarged to a larger, second cross-sectional area, thereby causing the overlapped region 16 to be removed or unfolded. The prosthesis 15 can move to expanded configuration when the retention sleeve 12 is in the deployed configuration. The prosthesis 15 is sized to expand within the body vessel so that the prosthesis 15 is engageable with the body vessel wall. In one example, the relative size between the enlarged passageway 46 of the retention sleeve 12 and the expanded prosthesis 15 can be substantially equal. In another example, the relative size between the expanded passageway of the retention sleeve and the expanded prosthesis can such that the expanded passageway is smaller than the expanded prosthesis. Here, the retention sleeve in the deployed configuration can be under tension due the expanded prosthesis.

In one aspect, the retention sleeve 12 may be attached to the prosthesis 12 at one or more attachment locations 82, as shown in the figures. This arrangement can ensure that the retention sleeve 12, having the integral sleeve or the pre-split sleeve configuration, remains attached to the prosthesis 15 during delivery and deployment of the prosthesis. In one example, the retention sleeve can be attached to the prosthesis at attachment locations by adhesives, sewing and tying a suture, stitching a suture, and/or forming tufts with suture materials. Various types of sutures may be used. For example, synthetic sutures may be made from polypropylene, nylon, polyamide, polyethylene, and polyesters such as polyethylene terephthalate. These materials may be used as monofilament suture strands, or as multifilament strands in a braided, twisted or other multifilament construction. Regardless of the type of suture employed, it is capable of being used to sew the retention sleeve to the prosthesis.

In another aspect, the retention sleeve 12, having either the integral sleeve or the pre-split sleeve configuration, may remain unattached to the prosthesis 15 so that the retention sleeve 12 is removable from the prosthesis after expansion of the prosthesis. This arrangement can allow for sealing and/or anchoring directly between the vessel wall and the expanded prosthesis, and may inhibit the possibility of leakage through the retentions sleeve. FIG. 4 depicts the retention sleeve 12 that can be peeled away from the prosthesis 15 when in the expanded configuration. For example, a pull tab 75 can be coupled to each of the first and second sleeve ends and can be extended toward the middle M of the prosthesis. The pull tabs 75 can be retracted away from the ends toward the middle of the prosthesis in the outside-in direction so that the pull tab splits open the retention sleeve along a split line.

In one example, the retention sleeve 12, such as, e.g., having the integral configuration, can be splittable for removal from the prosthesis after expansion. The retention sleeve can be splittable by use of any well-known means or material that permits the sleeve to be separated, preferably longitudinally, along a relatively predictable path. The retention sleeve is usually, but not necessarily separated into two or more portions, thereby opening a fissure along the length that permits its removal from around the prosthesis situated therein. A predetermined split line may be formed in the retention sleeve through which the tear or split progresses due to properties of, and/or features incorporated into the sleeve material. The means for splitting the retention sleeve can withstand being subjected to a curve to the degree required by the particular application without kinking or premature separation. The retention sleeve may include a splittable polymer such as molecularly oriented, non-isotropic PTFE that is used to make the PEEL-AWAY® Introducer Sheath (Cook Incorporated, Bloomington, Ind.), which is described in, e.g., U.S. Pat. No. 4,306,562 to Osborne and U.S. Pat. No. 4,581,025 to Timmermans, each of which is incorporated herein by reference in its entirety. The split line can be enhanced by adding at least one preweakened feature, such as a score line, perforations, or reduced wall thickness regions, extending longitudinally along the length of the sleeve. The longitudinal preweakened feature may be included anywhere from one or more orthogonal predetermined split lines to a helical type arrangement that may comprise only a single predetermined split line. The preweakened feature may have sleeve portions that engage each other with a zipper-like or tongue-and-groove-like interface, or any other splittable connection interface along the contacting lateral edges of the sleeve portion. Other examples of splittable sleeve configurations can be found in U.S. Pat. No. 6,447,540 to Fontaine et al. and U.S. Pat. No. 6,827,731, each of which is incorporated herein by reference in its entirety. The retention sleeve can have more than one split lines.

FIG. 5 depicts another example of the system having a plurality of overlapped regions 16A, 16B, 16C formed in the retention sleeve 12. Each overlapped region can have a corresponding releasable member 20A, 20B, 20C therethrough, respectively. It can be appreciated by those skilled in the art that two overlapped region or four or more overlapped regions can be similarly formed. The additional releasable members can distribute the retention forces circumferentially along the retention sleeve so that the forces are not concentrated along one location. This arrangement may allow for smaller folds at each location, as well as smaller sizes of releasable members since the wires no longer has to withstand such greater forces. All three releasable members may be removed at once to allow for full circumferential expansion of the prosthesis 15 at once. Alternatively, individual releasable members can be removed to allow for selective expansion of different circumferential segments of the prosthesis.

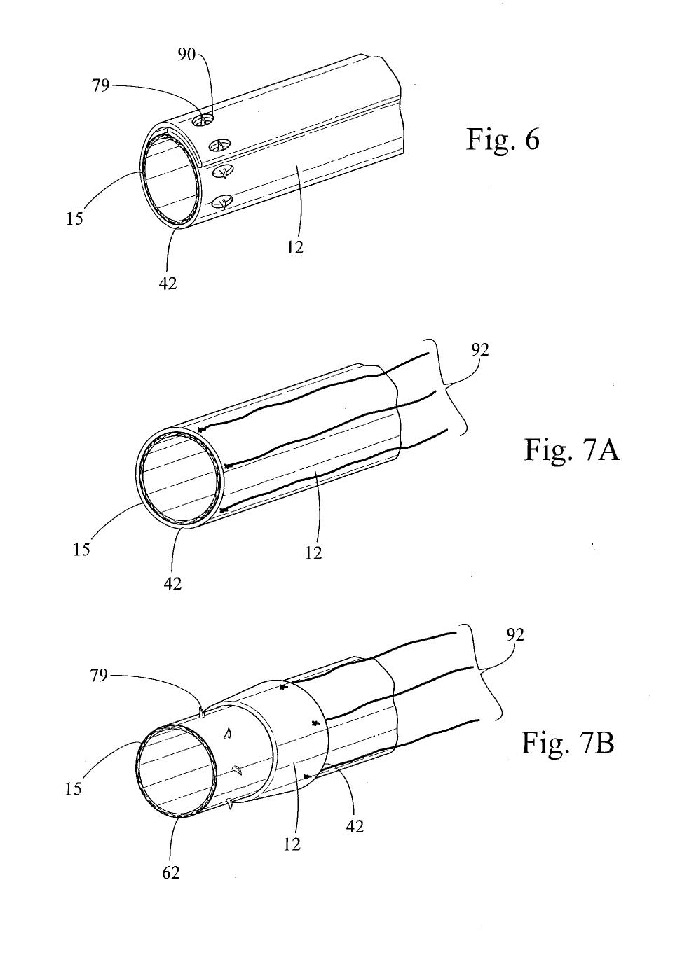

The prosthesis 15 can include a generally tubular graft body and a support structure together defining a fluid passageway. Although the prosthesis can be balloon expandable, it is preferred that the prosthesis is self-expandable. The support structure can be attached to the graft body by sutures sewn therein, wire, staples, clips, bonding agents, or other methods that may be used to achieve a secure attachment to the graft body. The prosthesis has a size and shape suitable for at least partial placement within a body vessel, such as an artery or vein, and most particularly, for placement at the site of a vascular trauma. The prosthesis may be easily manipulated during delivery to a transected artery or vein during emergency surgery, and particularly, to obtain hemostasis while maintaining blood perfusion. The support structure can be any stent pattern known to one skilled in the art. Examples of stent patterns is the Z-STENT® and ZILVER® stent, each available from Cook Medical (Bloomington, Ind.). An anchoring member 79 (see, e.g., FIG. 6) can be attached to or integrally formed with the support structure of the prosthesis 15 to provide vessel fixation and inhibit prosthesis migration. The anchoring member 79 can be configured to avoid adverse conditions associated with disturbing the vasa vasorum and/or pressure induced necrosis of the medium muscular arteries of the type that may result from tying ligatures circumferentially around a connector or a vascular conduit. The anchoring member 79 can include various shaped member structures, including barbs, fibers, bristles, or outer protruding and penetrable media. The anchoring member and/or support structure can be formed of a biocompatible metal, such as stainless steel (e.g., 316L SS), titanium, tantalum, nitinol or other shape memory materials, or a high-strength polymer.

The graft body can be formed from conventional materials well known in the medical arts. The graft body may comprise an expanded polytetrafluoroethylene (ePTFE), polytetrafluoroethylene, silicone, polyurethane, polyamide (nylon), as well as other flexible biocompatible materials. The graft body can also be formed from known fabric graft materials such as woven polyester (e.g. DACRON®), polyetherurethanes such as THORALON® from Thoratec Corporation (Pleasanton, Calif.), and polyethylene such as an ultra-high molecular weight polyethylene (UHMwPE), which is commercially available as DYNEEMA®. The graft body may also include a bioremodelable material, such as reconstituted or naturally-derived collagenous materials, extracellular matrix material (ECM), submucosa, renal capsule membrane, dermal collagen, dura mater, pericardium, fascia lata, serosa, peritoneum or basement membrane layers, intestinal submucosa, including small intestinal submucosa (SIS), stomach submucosa, urinary bladder submucosa, and uterine submucosa. One non-limiting example of a suitable remodelable material is the SURGISIS® BIODESIGN™, commercially available from Cook Medical (Bloomington, Ind.). Another suitable remodelable material is the graft prosthesis material described in U.S. Pat. No. 6,206,931 to Cook et al., which is incorporated herein by reference in its entirety.

Portions of the prosthesis can also include a coating of one or more therapeutic agents along a portion of the stent structure and/or the graft body. Therapeutic agents for use as biocompatible coatings are well known in the art. Non-limiting examples of suitable bio-active agents that may be applied to the vascular conduit include thrombo-resistant agents, antibiotic agents, anti-tumor agents, antiviral agents, anti-angiogenic agents, angiogenic agents, anti-mitotic agents, anti-inflammatory agents, angiostatin agents, endostatin agents, cell cycle regulating agents, genetic agents, including hormones such as estrogen, their homologs, derivatives, fragments, pharmaceutical salts and combinations thereof. Those skilled in the art will appreciate that other bioactive agents may be applied for a particular use. The bioactive agent can be incorporated into, or otherwise applied to, portions of the vascular conduit by any suitable method that permits adequate retention of the agent material and the effectiveness thereof for its intended purpose. Although the device has been described in connection with its primary intended use for repair of vascular trauma, those skilled in the art will appreciate that the device may also be used to repair other traumatic conditions. Non-limiting examples of such conditions include aneurysms, such as abdominal aorta aneurysms, and surgery for tumor removal.

The retention sleeve 12 can made of one or more biocompatible materials known in the art, such as, e.g., the graft body materials described herein. The materials selected for the retention sleeve preferably is strong enough to maintain its shape when in the delivery configuration. In other words, the retention sleeve can be configured not to stretch under tension provided by the compressed prosthesis. In addition, the openings 78 in the retention sleeve 12 can maintain their shape and orientation so not to stretch or deform under tension provided by the compressed prosthesis and with movement of the releasable member. It is contemplated that the openings can be reinforced to inhibit the possible of deformation, such as, e.g., with sutures sewn along the edge that defines each opening. Alternatively, grommets can be inserted thru the openings at predetermined locations in the graft as described in U.S. Pat. App. Publ. 2009/0149939 to Godlewski et al., which is incorporated herein by reference in its entirety. The retention sleeve 12 may made from a porous material to allow for spacing for the anchoring member 79 to extend therethrough more easily to anchor directly into the body vessel wall. In one example, when the retention sleeve is made from a woven fabric, the spacing between the weave pattern may be sized sufficiently to allow the anchoring member to pass therethrough. When the retention sleeve is a polymer tube, the tube may be sufficiently thin to permit the anchoring member to puncture the retention sleeve wall to directly anchor into the body vessel wall. Here, the anchoring member 89 may have a delivery profile where the anchoring member lies coplanar with the compressed prosthesis, and a deployed configuration, where the anchoring member extends outwardly beyond the surface of the prosthesis.

The releasable members 20, 22 can be made of one or more several biocompatible materials known in the art. Examples of releasable member materials include metal wires, such as stainless steel, copper, nitinol or other metals that are common in medical use. The wire material may be composed of suture materials such as described above. The releasable member may be further coated with a lubricious coating such as a hydrophilic coating or a fluoropolymer such as PTFE to increase the slidability between the releasable member and the retention sleeve.

FIG. 6 and FIGS. 7A-7B depict modifications to the retention sleeve to facilitate engagement between the vessel wall and the prosthesis. In FIG. 6, one or more apertures 90 can be formed in the wall of the retention sleeve to expose the anchoring member 79 of the prosthesis 15. The aperture 90 can be shaped and sized to permit one or more anchoring members to pass through for direct engagement with the body vessel wall. The aperture 90 can be formed at strategic locations along the circumference of the prosthesis to allow for engagement along the vessel wall.

In FIGS. 7A-7B, one or more pulling members 92 can be attached to the sleeve end 42 of the retention sleeve 12 and can be extended along the retention sleeve toward the middle of the prosthesis 15. When the pulling members 92 are retracted in a direction away from the outer end of the prosthesis, the sleeve end 42 can be everted along the prosthesis 15 to expose the outer end 62 of the prosthesis 15 to ensure engagement between the prosthesis and the vessel wall. In one example, removal of the sleeve end from the anchoring member 79 can facilitate contact with the vessel wall. The pulling members 92 can be made of the same material as the releasable member. In one example, the pulling members are sutures that are sewn to the end of the retention sleeve. The retention sleeve 12 can be everted by a small distance to a position to expose the anchoring member 79, or can be everted by a larger distance toward the middle of the prosthesis 15. At the middle of the prosthesis 15, the retention sleeve 12 can be cut away from the prosthesis and removed from the body.

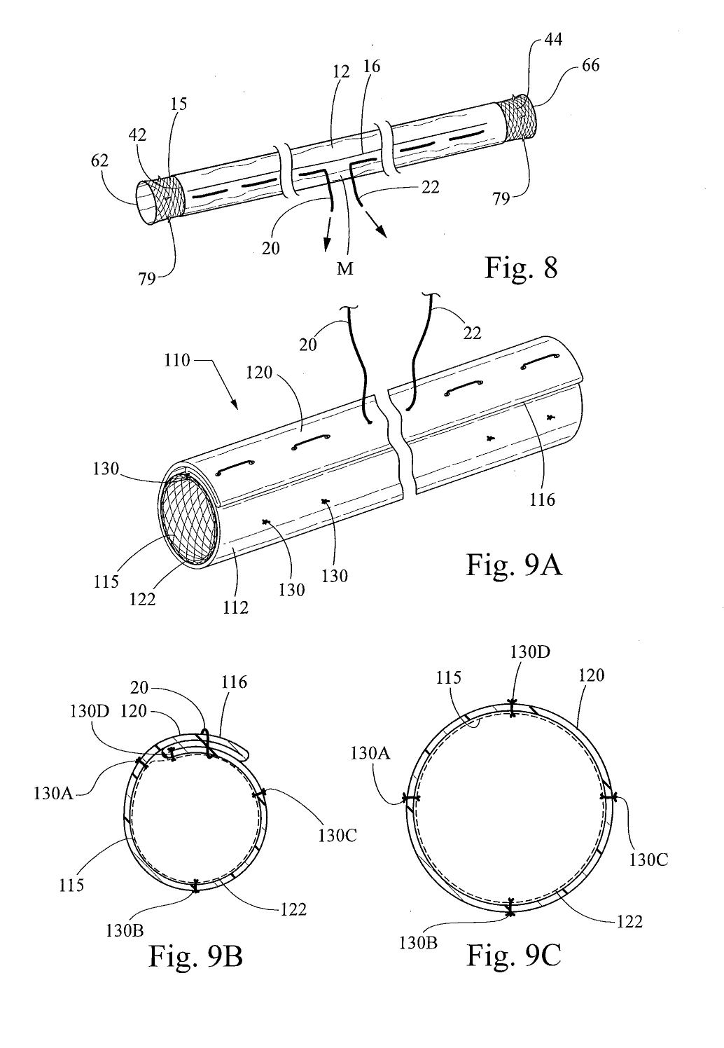

In FIG. 8, the length of the retention sleeve 12 can be shorter than the length of the prosthesis 15 such that the sleeve ends 42, 44 of the retention sleeve 12 terminate short of the outer ends 62, 66 of the prosthesis 15. As a result, the respective outer end portions of the prosthesis 15 remain exposed during the delivery process to ensure sufficient engagement between the prosthesis and the vessel wall. In one example, the exposed outer end portions can be provided with the anchoring member. It is contemplated that the exposed outer end portions of the prosthesis may be configured for minimal expansion when the retention sleeve is in place on a substantial intermediate portion of the prosthesis so that the end portions can fit within the vessel end openings. In one example, the support structure can include longitudinal struts that are arranged at the ends of the prosthesis to minimize expansion of the ends.

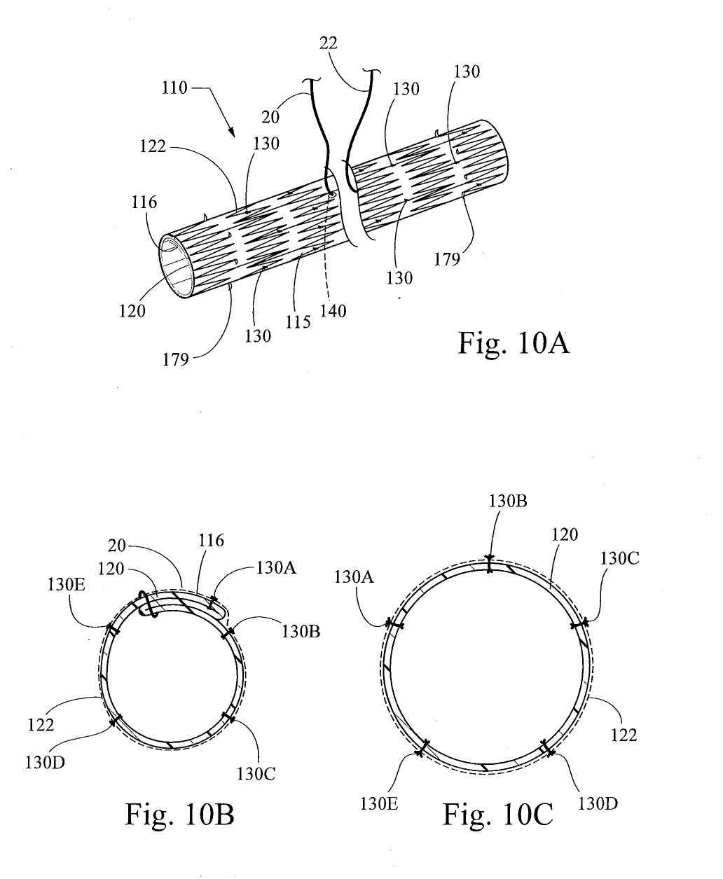

FIGS. 9A-10A depict other examples of a delivery system 110 where the graft body 120 of the prosthesis 115 forms the retention sleeve 112. Removal of an additional layer of material for the retention sleeve configuration, shown in, e.g., FIG. 1A, can reduce the overall delivery profile of the system. In this example, the retention sleeve 112 is integral with the graft body 120. In FIG. 9A, the graft body 120 can be attached along an exterior surface of the support structure 122 of the prosthesis at attachment regions 130. A sufficient amount of slackness in the graft body material can allow for the formation of the one or more overlapped regions 116. FIGS. 9B-9C are transverse sectional views showing the predetermined locations of the attachment regions, e.g., four attachment regions 130A, 130B, 130C, 130D. It is contemplated that the region of the graft body that defines the overlapped region remains unattached to the support structure 122 (shown in dashed lines) in order to form the overlapped region 116 when the support structure is in the compressed configuration and the graft body is in the delivery configuration as shown in FIG. 9B. In this example, a pair of attachment regions 130A, 130D can be brought closer in proximity to one another than with the remaining attachment regions so that the graft body 120 forms the overlapped region 116 and the support structure is radially compressed. Removal of the releasable members 20, 22 from the overlapped region 116 can permit radial expansion of the support structure 122 to the expanded configuration and movement of the graft body to the deployed configuration as shown in FIG. 9C.

In FIG. 10A, the delivery system 110 can include the graft body 120 attached along an interior surface of the support structure 122 of the prosthesis at attachment regions 130 with a sufficient amount of slackness in the graft body material to allow for the formation of one or more inner overlapped regions 116. The graft body along the interior can permit immediate exposure of the anchoring members 179 when employed. The provision of the graft body along the interior surface can provide a smoother surface for fluid flow, whereas the support structure along the exterior can provide for improved engagement between the prosthesis and the vessel wall. In this example, the inner overlapped region 116 is formed within the prosthesis. The releasable member is extendable through the inner overlapped region 116 and can extend outward from the graft body through a spacing or gap 140 (shown in dashed lines) formed between struts of the support structure. FIGS. 10B-10C are transverse sectional views showing the predetermined locations of the attachment regions, e.g., five attachment regions 130A, 130B, 130C, 130D, 130E. It is contemplated that the region of the graft body that defines the overlapped region remains unattached to the support structure 122 (shown in dashed lines) in order to form the inner overlapped region 116 when the support structure is in the compressed configuration and the graft body is in the delivery configuration as shown in FIG. 10B. In this example, a pair of attachment regions 130A, 130B can be brought closer in proximity to one another than with the remaining attachment regions so that the graft body 120 forms the inner overlapped region 116 and the support structure is radially compressed. Removal of the releasable members 20, 22 from the inner overlapped region 116 can permit radial expansion of the support structure 122 to the expanded configuration and movement of the graft body to the deployed configuration as shown in FIG. 10C.

The graft body 120 may be configured to fill in the voids or spacing between the strut members in order to have a substantially smooth luminal surface and/or a smooth outer surface. A prosthesis with a smooth luminal surface can allow the blood to flow more effectively and prevent blood from pooling within the prosthesis. A prosthesis with a smooth outer surface can provide a more effective sealing surface between the prosthesis and the vessel wall. In one example, the graft body can be a foamed material, such as an open-cell foam or another suitable biocompatible material (e.g., a lyophilized or sponge-form collagen material such as SIS), or expanded polytetrafluoroethylene (ePTFE). The foam material is spongy such that the support structure can be impressed into the foam material and the foam material surrounding the impressed support structure fills the voids between the strut members. The amount of desired penetration of the foam material within the voids to provide a smooth surface can depend on the degree of sponginess of the foam material, the strut width, and the percentage of support structure coverage of the graft body. In another example, the inner surface of the graft body may be modified with a recessed pattern of the support structure. When in the expanded configuration, the support structure can fit within the recessed region and the regions of the graft body outside the recessed region can fits within the voids to provide the smooth surface. The recessed pattern can be formed into the graft body with a masking and chemical etching process, a laser or water jet to mill or remove layers from the graft body, or any other process known in the art.

It is further contemplated that the retention sleeve and/or the graft body is expandable. For instance, the retention sleeve and/or the graft body can have shape memory characteristics such that the retention sleeve and/or the graft body has a compressed configuration below a threshold temperature, such as, e.g., the temperature of the patient's body, and an expanded configuration above the threshold temperature. One advantage of this configuration is the potential reduction of creases or folds in the expanded retention sleeve and/or graft body when in the expanded configuration, and thus the reduced risk of leakage via the creases. The retention sleeve and/or the graft body with shape memory characteristics also can facilitate the expansion of the support structure. To this end, the retention sleeve and/or graft body may include a woven fabric with shape memory element strands and/or textile strands in a first direction and a second direction. Suitable shape memory metals include, for example, TiNi (Nitinol), CuZnAl, and FeNiAl alloys, and particularly preferred are “superelastic” metal alloys. Superelasticity refers to a shape memory metal alloy's ability to spring back to its austenitic form from a stress-induced martensite at temperatures above austenite finish temperature. The austenite finish temperature refers to the temperature at which the transformation of a shape memory metal from the martensitic phase to the austenitic phase completes. One example of such retention sleeve with shape memory characteristics is described in U.S. Pat. App. Publ. 2008/0228028 to Carlson et al., which is incorporated herein by reference in its entirety.

Although the system has been described in connection with its primary intended use for repair of vascular trauma, those skilled in the art will appreciate that the system may also be used to repair other traumatic conditions. Non-limiting examples of such conditions include aneurysms, such as abdominal aorta aneurysms, and surgery for tumor removal.

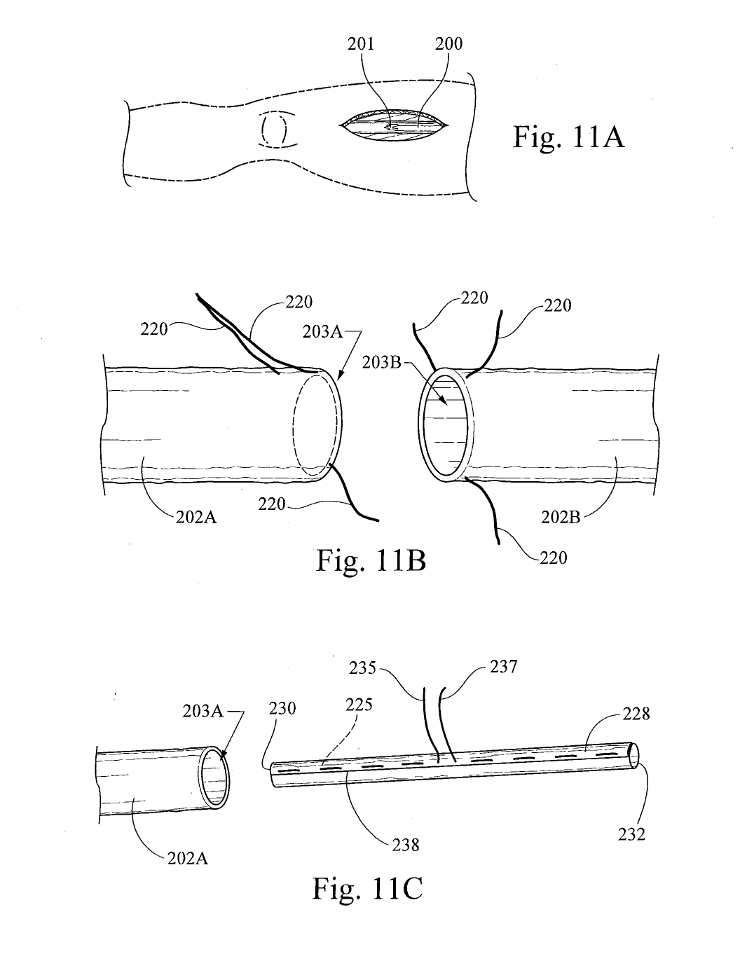

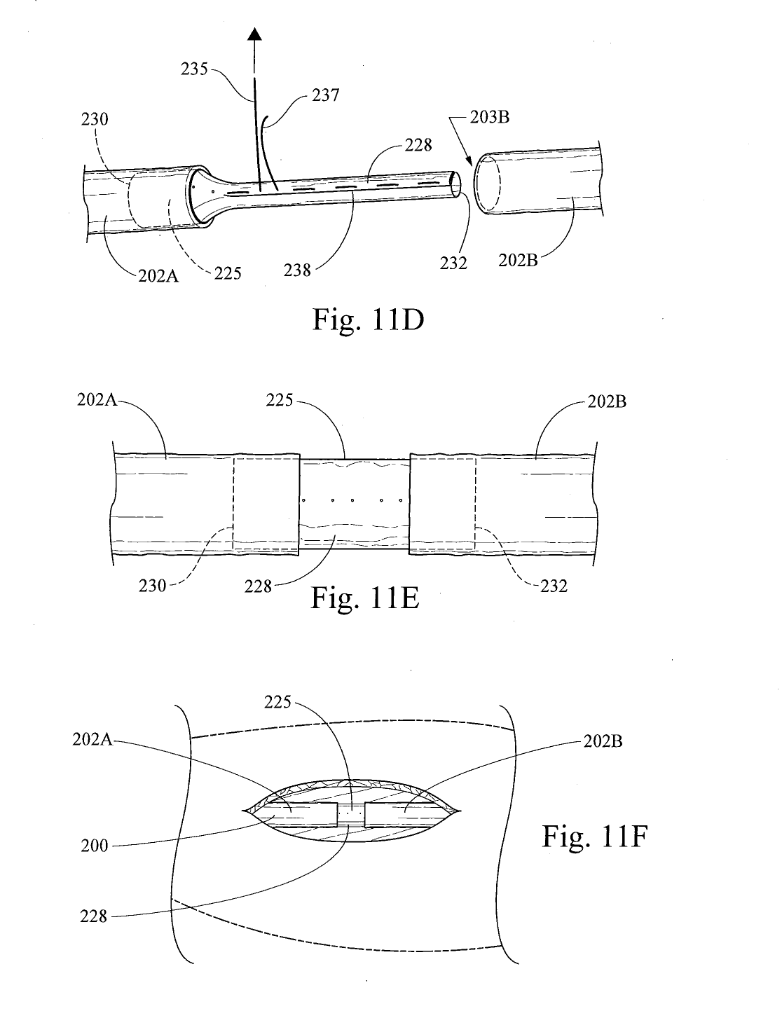

FIGS. 11A-11F illustrate a method of repair of a body vessel 200, such as open surgical repair of a body vessel. The body vessel 200 may be found, for example, in the leg of a patient. The body vessel 200 may have been subjected to a traumatic episode resulting in a portion 201 of body vessel 200 being torn away or otherwise severely damaged, as shown in FIG. 11A. Although the device can be inserted within the lacerated portion 201, it is typical to transect the body vessel in order to remove unhealthy vessel portions. Pre-surgery preparation may be applied to the leg, and a trauma pathway may be formed therein for open surgical access to the body vessel 200 and the damaged portion 201 thereof. After clamping the body vessel 200 on both ends of the damaged portion 201 with hemostats to restrict blood flow temporarily, the body vessel 200 can be cut or transected by the clinician into two portions 202A, 202B, as shown in FIG. 11B. The transection may be at the damaged portion 201 of the body vessel 200 or as far away as necessary from the damaged portion 201 to remove unhealthy portions of the body vessel 200 or unrepairable portions of the body vessel 200. Sutures 220 can be attached to the end openings 203A, 203B of the respective body vessel portions 202A, 202B to keep them fixed in place and open to facilitate insertion of the prostheses. Forceps may also be used in a similar manner. Any number of sutures 220 can be used to retain the end openings 203A, 203B in the open position, although triangulation sutures can be sufficient, with each suture being about 120 degrees apart from the adjacent suture. The prosthesis can be selected to have a radial expanded cross-section and a longitudinal length sufficient to bridge the body vessel portions 202A, 202B and fit radially within the body vessel portions.

FIG. 11C depicts the first end 230 and the second end 232 of the prosthesis 225 held in the radially compressed, delivery configuration by the retention sleeve 228. It can be appreciated by those skilled in the art that operation of the delivery system, similar to the delivery system 10 of FIG. 1, is for illustrative purposes only and that any other delivery system described herein is similarly operable. The first and second releasable members 235, 237 are shown coupled to the overlapped region 238. The first outer end 230 of the prosthesis 225 can be inserted through the end opening 203A of the vessel portion 202A by a sufficient distance for the purposes of engagement and/or anchoring.

In FIG. 11D, after insertion, a first portion of the prosthesis 225 can be permitted to expand from the delivery configuration to the deployed configuration. Such expansion may be initiated by removal or retraction of the first releasable member 235 from the overlapped region 238 in the direction of the arrow. FIG. 11D shows the first releasable member partially withdrawn. This can permit engagement of the first outer end 230 of the prosthesis 225 along the wall of the vessel portion 202A, or anchoring into the vessel wall when the anchoring members are employed. The second outer end 232 of the prosthesis 225 can remain outside of vessel portion 202A in the space between vessel portions 202A, 202B. The second outer end 232 of the prosthesis 225 can be inserted through the end opening 203B of the vessel portion 202B by a sufficient distance for the purposes of engagement and/or anchoring. After insertion, a second portion of the prosthesis 225 can be permitted to expand from the delivery configuration to the deployed configuration. Such expansion may be initiated by removal or retraction of the second releasable member 237 from the overlapped region 238. This can permit purchase of the second end 232 of the prosthesis 225 along the wall of the vessel portion 202B.

FIG. 11E depicts the expansion of the first outer end 230 and the second outer end 232 of the prosthesis 225 along the wall of vessel portions 202A, 202B. When present, the anchoring member (not shown) can engage the wall of vessel portions 202A, 202B to fix the respective outer ends of the prosthesis portion in place relative to the corresponding vessel portions. The retention sleeve 228 can remain in place after expansion of the prosthesis. Optionally, the retention sleeve can be removed, such as by splitting and peeling away the retention sleeve from the prosthesis such as described herein.

Portions of the exterior surfaces of the prosthesis end portions can sealably engage with the luminal walls of the body vessel to inhibit leakage of blood and to force blood to flow throughout the body vessel during emergency surgery, and particularly to obtain hemostasis while maintaining blood perfusion. FIG. 11F shows the prosthesis 225 deployed and interconnecting body vessel portions 202A, 202B within the leg of the patient. The prosthesis 225 can be adapted for permanent placement within the patient, thereby obviating a need for subsequent surgical intervention.

It can be appreciated by those skilled in the art that specific features of each embodiment of the deployment device are interchangeable among the device embodiments, even where no references to the specific features are made.

Drawings in the figures illustrating various embodiments are not necessarily to scale. Some drawings may have certain details magnified for emphasis, and any different numbers or proportions of parts should not be read as limiting, unless so-designated in the present disclosure. Those of skill in the art will appreciate that embodiments not expressly illustrated herein may be practiced within the scope of the present invention, including those features described herein for different embodiments, and may be combined with each other and/or with currently-known or future-developed technologies while remaining within the scope of the claims presented here. It is therefore intended that the foregoing detailed description be regarded as illustrative rather than limiting. And it should be understood that the following claims, including all equivalents, are intended to define the spirit and scope of this invention.

I claim:

1. A system for open surgical repair of a body vessel having a first vessel portion and a second vessel portion, the system comprising:
a prosthesis movable between a compressed configuration and an expanded configuration;
a sleeve member conformable into a tubular body having a passageway extending therethrough for receiving the prosthesis, the sleeve member movable between a delivery configuration and a deployed configuration, wherein in the delivery configuration the sleeve member has an overlapped region and the passageway has a first cross-sectional area sized to retain the prosthesis in the compressed configuration for insertion into a body vessel, and in the deployed configuration the passageway has a second cross-sectional area greater than the first cross-sectional area to allow for expansion of the prosthesis to the expanded configuration for engagement with the body vessel; and
a releasable member extending through the overlapped region of the sleeve member to retain the sleeve member in the delivery configuration, the releasable member being removable from the overlapped region to permit the sleeve member to move to the deployed configuration.

2. The system of claim 1, wherein the prosthesis has a first outer end, a second outer end, and an intermediate region therebetween, the releasable member is extended through the overlapped region from one of the outer ends to the intermediate region so that the releasable member is removed from the overlapped region in an outside-in direction to allow for expansion of the respective outer end of the prosthesis prior to the intermediate region of the prosthesis.

3. The system of claim 2, wherein the releasable member comprises a first releasable wire and a second releasable wire, the first releasable wire extended through a first length of the overlapped region defined between the first outer end and the intermediate region, the second releasable wire extended through a second length of the overlapped region defined between the second outer end and the intermediate region, wherein each of the releasable wires are removable from the respective lengths of the overlapped region in an outside-in direction to allow for expansion of the corresponding outer ends of the prosthesis.

4. The system of claim 1, wherein in the delivery configuration the sleeve member has a plurality of overlapped regions and a plurality of corresponding releasable members.

5. The system of claim 1, wherein the sleeve member is attached to the prosthesis at one or more attachment regions.

6. The system of claim 1, wherein the sleeve member is splittable for removal from the prosthesis after expansion of the prosthesis.

7. The system of claim 1, wherein the sleeve member comprises one or more apertures formed in a wall of the sleeve member to expose portions of the prosthesis.

8. The system of claim 1, wherein the sleeve member comprises one or more pulling members attached to a sleeve end of the sleeve member, wherein said sleeve end of the sleeve member is moved away from an outer end of the prosthesis with retraction of the pulling member to expose portions of the prosthesis.

9. The system of claim 1, wherein the prosthesis comprises a tubular graft body and a support structure coupled to the graft body, wherein the graft body forms the sleeve member, the graft body being coupled to an exterior surface of the support structure at a plurality of attachment regions, the attachment regions arranged so that the overlapped region is formed in the graft body when the support structure is in a radially compressed configuration.

10. The system of claim 1, wherein the prosthesis comprises a tubular graft body and a support structure coupled to the graft body, wherein the graft body forms the sleeve member, the graft body being coupled to an interior surface of the support structure at a plurality of attachment regions, the attachment regions arranged so that an inner overlapped region is formed in the graft body within the support structure when the support structure is in a radially compressed configuration.

11. The system of claim 1, wherein in the delivery configuration the sleeve member has at least one fold that defines the overlapped region.

12. The system of claim 1, wherein in the delivery configuration the sleeve member has a first edge and a second edge that define the overlapped region.

13. A system for repair of a body vessel, the system comprising:
a prosthesis movable between a compressed configuration and an expanded configuration, wherein the prosthesis has a first outer end, a second outer end, and an intermediate region therebetween;
a retention sleeve conformable into a tubular body and a sleeve passageway extending therethrough for receiving the prosthesis, the retention sleeve movable between a first configuration and a second configuration, wherein in the first configuration the retention sleeve has at least one overlapped region and the sleeve passageway is sized to retain the prosthesis in the compressed configuration for insertion into a body vessel, and in the second configuration the sleeve passageway has a larger cross-sectional area to allow for expansion of the prosthesis to the expanded configuration for engagement with the body vessel;
a first releasable wire member extending through a first length of the overlapped region of the retention sleeve from the first outer end to the intermediate region; and
a second releasable wire member extending through a second length of the overlapped region of the retention sleeve from the second outer end to the intermediate region,
wherein in response to removal of the first releasable wire member from the first length of the overlapped region of the retention sleeve in a first outside-in direction at the intermediate region of the prosthesis, the first outer end of the prosthesis is allowed to expand, and in response to removal of the second releasable wire member from the second length of the overlapped region of the retention sleeve in a second outside-in direction, opposite the first outside-in direction, at the intermediate region of the prosthesis, the second outer end of the prosthesis is allowed to expand.

14. The system of claim 13, wherein the retention sleeve is configured to be removed from the prosthesis after expansion.

15. A method of open surgical repair of a body vessel having a first vessel portion and a second vessel portion, the method comprising:
providing a prosthesis retained in a compressed configuration by a sleeve member in an overlapped configuration, wherein the sleeve member is maintained in the overlapped configuration with a releasable member extending through an overlapped region;
introducing a first outer end of the prosthesis into a first vessel portion, the first outer end being retained in the compressed configuration by a first segment of the sleeve member in the overlapped configuration; and
removing the releasable member from the overlapped region of the sleeve member to permit movement of the first segment of the sleeve member to a larger cross-sectional area such that the first outer end of the prosthesis is permitted to expand for engagement with a wall of the first vessel portion.

16. The method of claim 15, further comprising introducing a second outer end of the prosthesis into a second vessel portion, the second outer end being retained in the compressed configuration by a second segment of the sleeve member in the overlapped configuration; and
removing the releasable member from the overlapped region of the sleeve member to permit movement of the second segment of the sleeve member to a larger cross-sectional area such that the second outer end of the prosthesis is permitted to expand for engagement with a wall of the second vessel portion.

17. The method of claim 16, wherein the first segment of the sleeve member is maintained in the overlapped configuration with a first releasable member extending through a first length of the overlapped region, and the second segment of the sleeve member is maintained in the overlapped configuration with a second releasable member extending through a second length of the overlapped region,
wherein the removing step further comprises removing the first releasable member from the first length of the overlapped region, and removing the second releasable member from the second length of the overlapped region, wherein the first and second releasable member steps are removed from an intermediate region of the prosthesis such that the first and second outer ends of the prosthesis are permitted to expand prior to the intermediate region.

18. The method of claim 15, further comprising removing the sleeve member from the prosthesis after expansion of the prosthesis.

19. The method of claim 15, further comprising moving a sleeve end of the sleeve member to expose the first outer end of the prosthesis to the corresponding portion of the vessel portion.

20. The method of claim 15, wherein the prosthesis comprises a tubular graft body and a support structure coupled to the graft body, wherein the graft body forms the retention sleeve, the graft body being coupled to a surface of the support structure at a plurality of attachment regions, the attachment regions arranged so that the overlapped region is formed in the graft body when the support structure is in a radially compressed configuration.
